(19) United States
(12) Patent Application Publication
(10) Pub. No.: US 2016/0356789 A1
(43) Pub. Date: Dec. 8, 2016

(54) Method of Detection of Summed Cardiac Troponins for Cardiovascular Risk Stratification

(71) Applicant: True Health Diagnostics LLC, Frisco, TX (US)

(72) Inventors: Rebecca E. Caffrey, N. Chesterfield, VA (US); Joseph P. McConnell, Richmond, VA (US); Mohmed E. Ashmaig, Richmond, VA (US)

(21) Appl. No.: 15/091,835

(22) Filed: Apr. 6, 2016

Related U.S. Application Data
(60) Provisional application No. 62/143,454, filed on Apr. 6, 2015.

(51) Int. Cl. G01N 33/68

(52) U.S. Cl. CPC G01N 33/6887; G01N 2333/4712; G01N 2800/32; G01N 2800/7019

(57) ABSTRACT

The present disclosure provides methods for assessing a subject's cardiovascular health. In one embodiment, the method comprises determining a level of cardiac Troponin I and a level of cardiac Troponin T in a sample, and comparing a combined score determined as a function of the cardiac Troponin levels to reference cardiac Troponin levels associated with a reference population. In some embodiments, the present disclosure provides methods of treating a subject, the method comprising determining a level of cardiac Troponin I and a level of cardiac Troponin T in a sample, comparing a combined score determined as a function of the cardiac Troponin levels to reference cardiac Troponin levels associated with a reference population, and initiating a therapeutic regimen in the subject if the combined score is greater than the reference cardiac Troponin levels.

### PRIORITY CLAIM

This application claims priority to U.S. Provisional Patent Application Ser. No. 62/143,454, filed Apr. 6, 2015, the entire contents of which are incorporated herein by reference and relied upon.

### BACKGROUND

Currently cardiac Troponin T and cardiac Troponin I are measured separately, depending on physician preference for one over the other, as biomarkers of prevalent MI or ACS when the levels of each, respectively, exceed the 99^{th} percentile Upper Reference Limit (URL) for a population. Recent advances in cTn assay technology has lowered detection limits for both cardiac Troponin I and cardiac Troponin T, enabling development of high sensitive (hs) assays for these analytes and earlier detection of adverse cardiac events in an acute care setting with symptomatic patients. However, we have observed in our unpublished data that there is a large disagreement between the different cardiac troponin assays (particularly cardiac Troponin I) produced by different manufacturers; these discrepancies are not well understood but it is thought that the discrepancies arise partly due to differences in assay design (e.g. specific epitopes of the given troponin recognized by both the capture and detection moieties, as well as differences in sensitivity of the detection technologies). However, significant biological variability in the detectability of cardiac Troponin T and cardiac Troponin I in the bloodstream also exists due to many factors, including the absolute amount of the cTn proteins present, the binding of the troponins to binding proteins, the amount of free vs. bound (complexed) proteins, which epitopes of the troponins are available (exposed) for binding by capture moieties such as cardiac troponin-specific antibodies, and the half life/degradation process of the cardiac troponins in the bloodstream. Additionally, while cardiac Troponin T and cardiac Troponin I levels generally increase in a similar trajectory with cardiac injury and various disease states, this is not always the case; a given sample may be positive for cardiac Troponin T might be negative for cardiac Troponin I, and vice versa. One study found 10% of patients had discordant results (one form of troponin elevated past URL but not the other) when cardiac troponin levels were compared to 30 day mortality; obviously if only one form of cardiac troponin was measured in these patients there would be many false negatives and patients at high risk would be missed. The end result of this biological and assay variability is lower assay sensitivity and a larger number of false negative test results when comparing an individual patient's cardiac Troponin T and cardiac Troponin I levels to the 99^{th} percentile URL for diagnosis of MI or ACS.

However, we have realized that these same phenomena confound interpretation of data within the “normal” range of cardiac Troponin T and cardiac Troponin I for the same reasons. This presents a clinical problem because there are many cardiodiabetic conditions and other disease states which can elevate one or both cardiac troponins slightly or moderately, but not enough to exceed the 99^{th} percentile URL. Current methods involve the analyses of either cardiac Troponin T or cardiac Troponin I, but not both. An assay that combines measurements for cardiac Troponin T and cardiac Troponin I together by utilizing multiple capture and detection antibodies for multiple epitopes of cardiac Troponin T and cardiac Troponin I in the same instance, and yielding a single measurement result comprised of total cardiac troponins, would enable more accurate detection of the “real” sum levels of cardiac troponins in a patient's blood. This would have utility and help to identify the patients at high risk for ACS, MI, and mortality as described above when the total troponin measurement is above the URL. But the combined measurement would also have clinical utility when the patient is not experiencing symptoms of an MI or ACS and is unlikely to have total cardiac troponins elevated over the URL. By combining capture and detection of both types of cardiac troponins in a single assay, the assay sensitivity will be improved over current technologies that measure the analytes separately. The true level of cardiac-specific troponins can be measured more accurately, with greater precision; LOQ will be lowered (improved) due to lower CVs, and there will be fewer false negatives. Therefore this new combinatorial total troponin assay will increase the diagnostic and prognostic utility of cardiac troponin measurements, particularly in the low/normal range of their expression and particularly in the absence of other biomarkers of cardiac muscle damage in common use such as BNP, NT proBNP, sST2, Galectin-3, GDF-15, CKMB and myoglobin. This combined troponin assay will enable reliable diagnosis, prognosis, monitoring, and therapy guidance in the range above the LOQ and below the 99^{th} percentile URL for a variety of disease states known to cause mild to moderate elevation in cardiac troponins. The assay components and method will be incorporated into a specific kit format.

### SUMMARY

In some embodiments, the present disclosure provides a method of treating or preventing a cardiovascular disease or disorder in a subject, the method comprising determining a cardiac Troponin I level and a cardiac Troponin T level in a biological sample of the subject; calculating an combined score as a function of at least the determined cardiac Troponin I level and the determined cardiac Troponin T level; comparing the combined score to reference values from a reference population; and initiating a treatment regimen in the subject if the combined score correlates with a range in a higher unit of an ordered distribution of the reference values.

In some embodiments, the present disclosure provides a solid surface comprising at least one capture antibody capable of binding to cardiac Troponin T and at least one capture antibody capable of binding to cardiac Troponin I.

### BRIEF DESCRIPTION OF THE FIGURES

FIG. 1 shows organization of the troponin, tropomyosin, and actin complex in relaxed (A) and contracting muscle (B). In the relaxed state, tropomyosin lies in the groove between the actin filaments. Following muscle stimulation, calcium binds to troponin and induces a conformational change in tropomyosin, exposing the myosin-binding sites on the actin filament so that muscle contraction can occur.

FIG. 2 illustrates biphasic release of cardiac Troponin T after AMI and that timing of rise/fall in cardiac Troponin T and cardiac Troponin I are similar in days after AMI but not identical.

FIG. 3 shows a range of cTn levels from high levels caused by cardiomyocyte necrosis in MI, to those reflective of ischemic states, and low levels that typify the normal physiological state.

FIG. 4A shows evolution of the cardiac Troponin I (cardiac Troponin I) assays and their diagnostic cutoffs. A hypothetical case of acute coronary syndrome is depicted with the earliest times of potential diagnosis corresponding to the diagnostic cutoffs of more sensitive cardiac Troponin I assays. The years correspond to the availability of the respective assays in the US market. Note that as high sensitivity assays have evolved, the LOD and LOQ have moved lower and lower, thus lowering cutoffs for the 99th percentile diagnostic cutoff decision limit.

FIG. 4B is a diagrammatic representation of levels of cardiac Troponin I in the 99th percentile reference population (top panel) and the ACS population (distribution by hours after symptom onset). Note overlap in cardiac Troponin I values between healthy reference population and acutely symptomatic ACS population.

### DETAILED DESCRIPTION

The current invention comprises the measurement of summed cardiac Troponin I (cardiac Troponin I) and cardiac Troponin T (cardiac Troponin T) levels in a subject, including subjects not suffering from acute coronary syndrome, for risk stratification of cardiovascular health and cardiodiabetes. Additive measurement of cardiac Troponin T and cardiac Troponin I may enable detection at very low concentration enabling more accurate prognostic information about the subject's long-term risk of ischemic event, heart failure, or indication of myocardial damage. A combined measurement of cardiac Troponin T and cardiac Troponin I captures the variety of patients with discordant cardiac Troponin T and cardiac Troponin I levels that otherwise would be overlooked by a single marker assay.

Cardiac troponin (cTn) is a complex of proteins expressed in cardiac muscle cells (cardiomyocytes) that is fundamental to the contraction of heart muscle. cTn is released into the bloodstream when cardiomyocyte necrosis occurs as a result of injury to the cardiac tissue. Increased blood levels of cTn are therefore used as a highly selective marker of cardiac damage. cTn has emerged as the preferred biomarker in the universal definition of myocardial infarction (MI) and has traditionally been used to “rule-in” or “rule-out” the occurrence of acute MI. The recent development of a new class of high sensitivity cTn (hs-cTn) assays has permitted the detection of very low circulating levels of cTn, potentially enabling use of low to moderate levels of hs-cardiac Troponin I and hs-cardiac Troponin T as effective tools for the prognostic evaluation and risk stratification of cardiovascular (CV)-related diseases. The measurement of hs-cTn allows for earlier identification of high-risk patients that may require more aggressive treatment strategies.

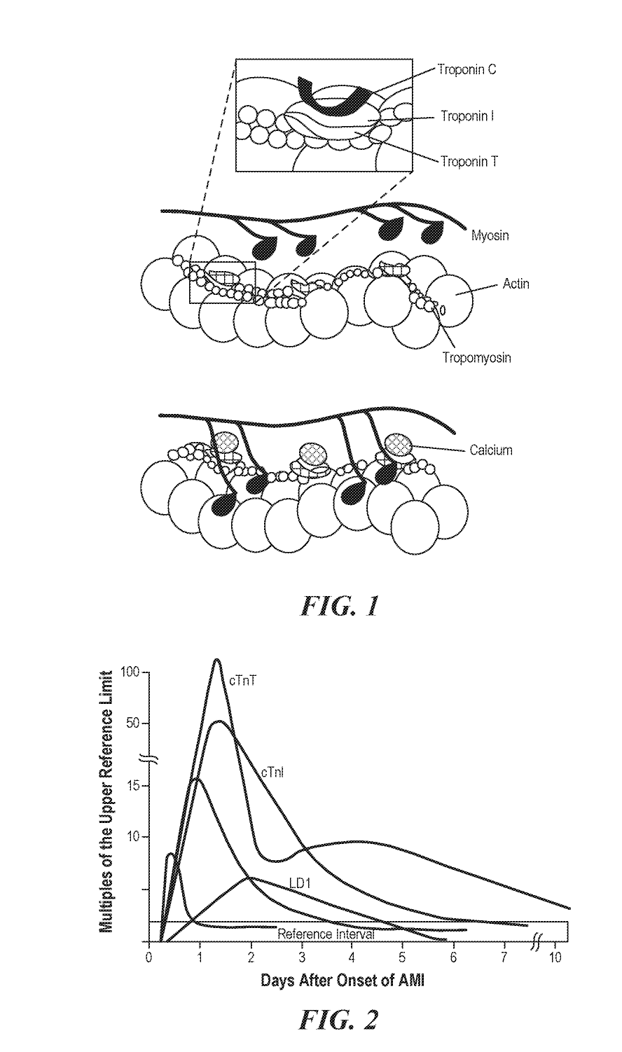

Following nervous stimulation of muscle tissue, contraction occurs from the coordinated action of several muscle proteins. Tropomyosin lies in the groove between actin filaments in striated (skeletal and cardiac) muscle and regulates the binding of myosin to actin (FIG. 1). In the resting state, tropomyosin prevents the formation of crossbridges between myosin and actin. Upon muscle stimulation, a conformational change in tropomyosin must occur in order to expose the myosin-binding sites upon the actin filaments. Troponin directs this conformational change.

Troponin is a heterotrimeric complex composed of T, I, and C subunits (FIG. 1 inset). All three of these polypeptide chains are named for their unique and essential functional properties. Troponin T (TnT) preserves the attachment of the troponin complex to tropomyosin. Troponin I (TnI) inhibits myosin from interacting with actin in the resting state. Stimulation of the muscle tissue induces the release of calcium ions, which then bind regulatory sites present upon troponin C (TnC). This induces the rapid conformational change in troponin that shifts the entire troponin/tropomyosin complex away from actin, exposing myosin-binding sites upon the actin filament. The formation of crossbridges between myosin and actin filaments (FIG. 1, bottom) allows the myosin heads to “walk” along the actin filaments and, using ATP, bend to pull past the actin, so shortening the sarcomere and contracting the muscle.

The structures of cardiac troponins I (cardiac Troponin I) and T (cardiac Troponin T) are distinct from the skeletal forms, making these useful and selective markers of cardiac damage, unlike cardiac Troponin C (cardiac Troponin C), which is identical to its skeletal counterpart. While the majority of cellular cTn, which collectively refers to all three cardiac troponins, is bound to tropomyosin and the actin filaments, an additional pool of cTn resides in a free, unbound form in the cytosol of the cell. This free cTn pool comprises 6-8% of cellular cardiac Troponin T and 2-4% of cellular cardiac Troponin I.

Acute coronary syndromes (ACS) such as MI cause substantial damage to the cardiomyocytes, leading to a characteristic, biphasic pattern of troponin release. An initial wave of cTn reaches a peak within 24-48 hours (FIG. 2), although it is detected as early as 2-4 hours after the onset of chest pain (FIG. 5). This early rise in ern is a consequence of unbound troponin in the free cytosolic pool leaking through the compromised cell membranes of injured cardiomyocytes into the blood. Following the initial cell damage, necrosis of cardiomyocytes and degradation of the actin filaments leads to a second release of structurally-bound troponin. This second wave causes a prolonged elevation of blood cardiac Troponin I levels that may persist for as long as 10-21 days following MI (not shown on diagrams).

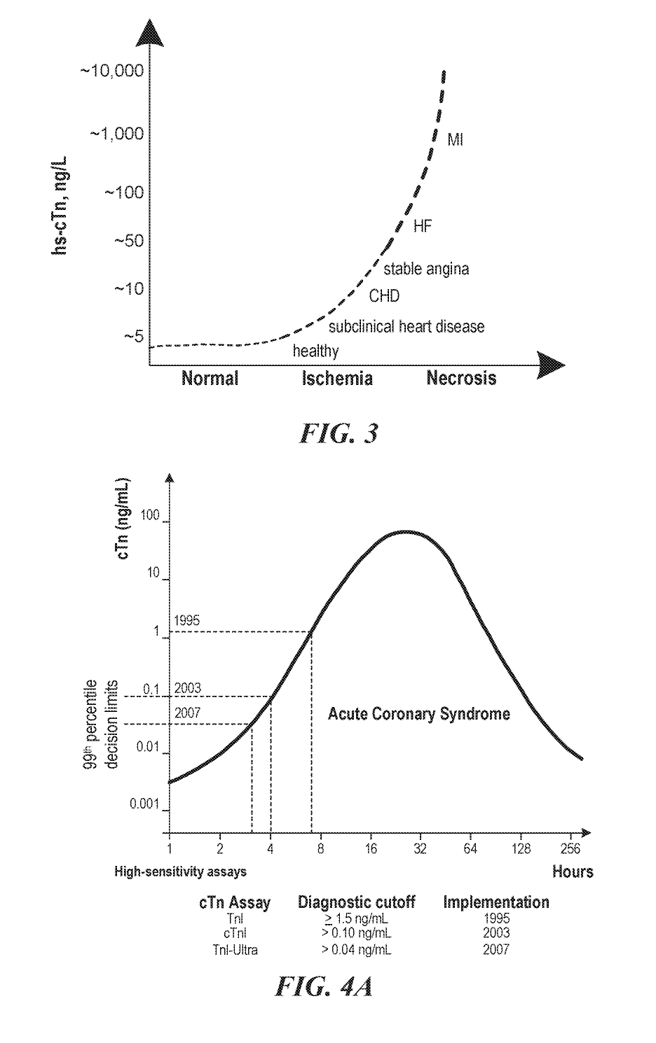

Newly-developed high-sensitivity assays for individual cardiac troponins allow earlier detection of rising cTn levels during an acute MI. The use of these assays has led to the finding that persistently elevated levels of hs-cTn are associated with chronic disease states such as coronary artery disease (CAD), type 2 diabetes mellitus (T2DM), and heart failure (HF). Unlike in MI, where extremely high levels of cTn are detected following massive cardiomyocyte necrosis, the levels of cTn detected in chronic disease conditions are more moderate and are thought to be associated with micronecrosis, ischemia, or reversible cardiomyocyte injury (FIG. 3). These events may be asymptomatic; indeed, in patients with ischemic heart disease, as many as 75% of acute episodes of myocardial ischemia are estimated to be asymptomatic, and considered “silent”. Myocardial cell damage and/or cell death, and subsequent release of hs-cTn, may be caused by increased left-ventricular (LV) wall stress associated with ventricular chamber dilation and wall thinning, epicardial CAD, neurohormonal activation, inflammatory cytokines, altered calcium handling, and oxidative stress. Low levels of hs-cTn are also detected in patients undergoing stress testing, suggesting that even transient ischemic events can induce cTn release in the absence of necrosis, perhaps independent of reversible myocardial ischemia. High sensitivity assays have also facilitated the measurement of detectable levels of hs-cTn in the blood of healthy individuals, the presence of which suggests a natural process of cardiomyocyte turnover that is characteristic of the normal physiological state (FIG. 3).

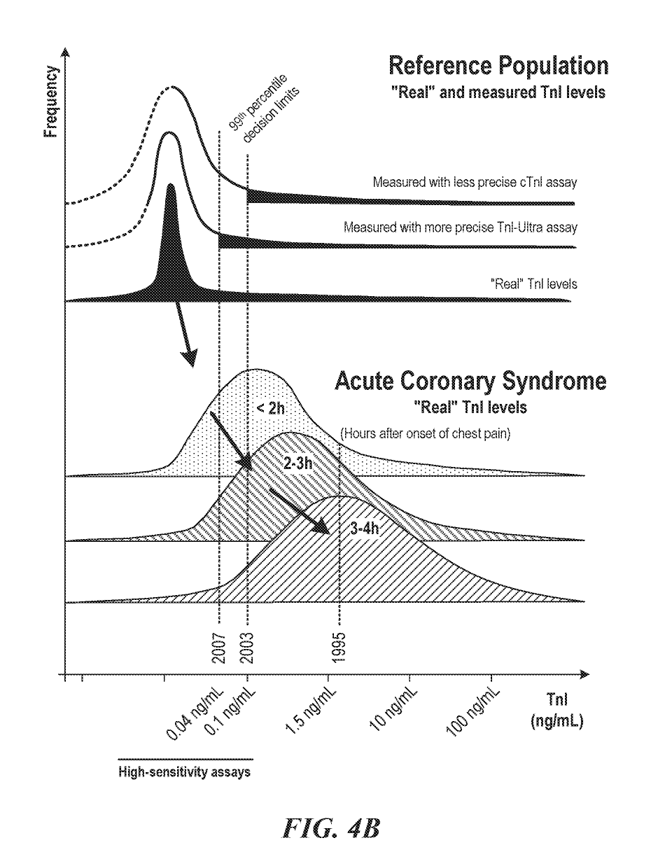

In summary, the enhanced sensitivity of the hs-cTn assays expands the clinical use of this prognostic and diagnostic marker to asymptomatic patients. High sensitivity cardiac Troponin I concentrations of <0.07 ng/mL or below and high sensitivity concentrations of hs-cardiac Troponin T below <0.1 ng/mL are considered to be in an optimal, healthy range. Levels of either cTn above the 99^{th} percentile URL are associated with high risk of adverse outcomes including CV events, HF, or death. Due to improvements in assay technology in recent years, the 99^{th} percentile URL has been a moving target (see FIG. 4), and controversy exists over where to draw the cutoff in view of the constant improvement in detection in low concentrations. Either independently, or in combination with other markers of cardiac damage, the levels of hs-cardiac Troponin I and hs-TnT do provide an effective tool for risk stratification and identification for most patients at high risk of MI, ACS, and mortality for the selection of more aggressive risk-reduction strategies.

cardiac Troponin T and cardiac Troponin I have been particularly useful in the acute care setting to differentiate patients with actual myocardial infarction from patients who are not in cardiac distress but with similar symptoms. High levels of cardiac Troponin T or cardiac Troponin I (99^{th} percentile URL) are more specific to cardiac injury than other biomarkers such as CKMB and myoglobin, and testing for these in an acute care setting enables prompt emergency treatment for AMI. In a conventional cardiac Troponin T assay such as the Roche Cobas® Troponin T hs 99^{th} percentile threshold is 0.14 μg/L and the 99^{th} percentile for hsTnT is 0.0141.μg/L (14 pg/ml). Increases in assay sensitivity have generated confusion and adjustment to use for this and more prognostic purposes. At least one technology and assay type exists to measure troponin concentration as low as 3 pg/ml. Thus the dynamic range of cardiac troponin detection capability now spans several orders of magnitude.

In a 1998 study, Christenson et al (Clinical Chemistry 1998, incorporated herein in its entirety) studied the different predictive abilities of cardiac Troponin I and cardiac Troponin T in patients that present to medical facilities within a spectrum of acute coronary syndromes. Their investigation focused on those critical care patients meeting a 99^{th} percentile threshold for either cardiac Troponin I or cardiac Troponin T. They found that while increased concentration of cardiac Troponin T and cardiac Troponin I was independently associated with greater risk of mortality 30 days after presentation, 10% of the patients were showed discordant results, wherein only one of the cardiac troponins were elevated into the threshold defining higher risk for additional cardiac events. Thus while individual measurements of either cardiac troponin are useful, a significant proportion of patients, even in the highest risk categories with presumably the highest levels of both troponins, will be missed if only one form of cardiac Troponin Is measured. An assay that simultaneously measures both forms and utilizing capture and detection antibodies with multiple epitope specificities should increase accuracy of cTn measurements and thus enable detection of more true positives and more accurate assignment of risk.

There is significant biological variability in cardiac Troponin I and cardiac Troponin T within individuals on serial measurements, and this biological variation is particularly significant when measuring in the lower range of troponin concentrations. One approach to this problem has been calculation of Reference Change Values (RCV's) which involves measuring the cTn concentration at 2 different timepoints, then performing a calculation with terms representing biological variability and analytical (assay) variability. Biological variability can also be expressed as the index of individuality (II), which is calculated as (within subject variation/between subject variation). For cardiac Troponin I, the within subject variation is higher than the variation between subjects for both short-term and long-term measurements. For cardiac Troponin T, the short- and long-term II for cardiac Troponin T were higher. Examples of causes of variability in cardiac Troponin T in the low range would be intense exercise (i.e. marathon running), renal disease, diabetes, sleep apnea, and smoking. Moderate exercise may lower cardiac Troponin T. cardiac Troponin I may also be affected by diet and lifestyle, for example, a Mediterranean-style diet has been shown to lower cardiac Troponin I in the low ranges.

The physiological complexity of circulating cardiac troponins is a confounder for accurate measurement by existing assays, and particularly for hs-cardiac Troponin I assays which vary far more than the Roche assay which dominates the market. Although both cardiac-specific troponins may be used to detect myocardial ischemia, they have distinct physical properties. For example, cardiac Troponin I (201 amino acids) is smaller and more hydrophobic than cardiac Troponin T (298 amino acids). In addition, cardiac Troponin T chiefly exists as a free protein in the blood, while cardiac Troponin I primarily circulates bound to cardiac Troponin C. Both proteins circulate at very low levels as the intact ternary complex of cardiac Troponin I-cardiac Troponin T-cardiac Troponin C. Furthermore, cardiac Troponin T and cardiac Troponin I may be altered by phosphorylation or proteolytic degradation, modifications that potentially mask or degrade regions of the protein recognized by cardiac Troponin I or cardiac Troponin T antibodies and impede antibody detection. Variation in the circulating forms of troponins—especially their binding partners and modifications—may affect antibody detection of specific epitopes. This supports our claim that an heterogeneous assay utilizing more than one detection pull-down antibody would yield a more accurate measurement of the cardiac troponins, particularly in the low ranges near the LOD and LOQ that occur in an apparently healthy population.

Taken together, the different physical properties of cardiac Troponin T and cardiac Troponin I, their potential for modification and degradation, as well as the variation in clinical assays strongly suggests that hs-cardiac Troponin I and hs-cardiac Troponin T levels are often related but not directly comparable. This premise is supported by several investigations including the PEACE study of participants with stable CAD, where hs-cardiac Troponin I predicted CV risk independently of traditional risk factors and hs-cardiac Troponin T (which has been shown to be a risk factor by many other studies), suggesting that hs-cardiac Troponin I and hs-cardiac Troponin T do not provide identical risk information for all forms of cardiovascular disease. There is no evidence to suggest the clinical advantage of measuring hs-cardiac Troponin T versus hs-cardiac Troponin I; indeed, meta-analysis of the prognostic use of cTn for the diagnosis of MI concluded that the markers provide similar information. Some studies have shown that a greater percentage of the population have detectable hs-cardiac Troponin I levels compared to hs-cardiac Troponin T levels, and these observations could be due to differences in age of study participants, assay sensitivity, technical differences, different patterns of cardiac Troponin T and cardiac Troponin I release, degradation, or stability, and even heterogeneity in study populations. Taken together, the heterogeneity of circulating forms of cTn, the biological variability within and between subjects, and clinical studies suggesting that each cardiac troponin may measure closely related but not identical aspects of risk of cardiovascular disease or cardiodiabetes supports our claim that an additive cTn measurement by heterogeneous assay would be a more accurate assessment of risk.

Serial troponin testing is not practical in an outpatient, non-critical care setting to assess or overcome variability in individual troponins due to their respective biological and analytical causes. We believe that a way to meaningfully reduce variability, increase measurement accuracy, and hence enable more accurate determination of risk would be to measure both types of troponin in the same assay, by means of multiple capture and detection antibodies (i.e. a heterogenous assay).

In some embodiments, the present disclosure provides at least one capture antibody capable of binding (e.g., selectively binding) a form of cardiac Troponin T. In some embodiments, the form of cardiac Troponin T comprises whole free cardiac Troponin T, proteolytic fragment of cardiac Troponin T, and/or a post-translational modification of cardiac Troponin T (e.g., phosphorylated cardiac Troponin T).

In some embodiments, the present disclosure provides at least one capture antibody capable of binding (e.g., selectively binding) a form of a non-cardiac Troponin T cardiac troponin. In some embodiments, the form of the non-cardiac Troponin T cardiac Troponin Comprises whole free cardiac Troponin I, an epitope of cardiac Troponin I which is exposed when it is bound in complex to cardiac Troponin C, an epitope of cardiac Troponin C that is exposed when it is bound in complex to cardiac Troponin I, an epitope unique to a bound cardiac Troponin I:cardiac Troponin C complex, a proteolytic fragment of cardiac Troponin I, and/or a post-translational modification of cardiac Troponin I (e.g., phosphorylated cardiac Troponin I).

In some embodiments, the present disclosure provides a solid surface comprising a plurality of capture antibodies. In some embodiments, the solid surface comprises magnetic/latex beads. In other embodiments, the solid surface is a micro-titer plate.

In some embodiments, a biological sample from a subject is applied to the capture antibodies (e.g., to a solid surface comprising capture antibodies). In some embodiments, the capture antibodies are washed after allowing the biological sample to remain in contact with the capture antibodies for a period of time sufficient to permit the capture antibodies to bind to antigens (e.g., cardiac troponins) present in the biological sample.

In some embodiments, methods of the present disclosure utilize a plurality of capture antibodies for capturing a plurality of forms of cardiac troponin. In some embodiments, the plurality of capture antibodies comprises at least one capture antibody capable of binding a form of cardiac Troponin T, at least one capture antibody capable of binding a form of cardiac Troponin I, and optionally at least one capture antibody capable of binding a form of cardiac Troponin C and/or a cardiac Troponin C:cardiac Troponin I complex.

In some embodiments, a capture antibody disclosed herein may be labelled with a detection tracer. Suitable detection tracers are those known in the art including, but not limited to, Horseradish peroxidase, chemiluminescence tracers, fluorescence tracers, and electrochemiluminescence tracers. In some embodiments, the detection tracers are detected by a detector for quantifying an amount of a cardiac troponin bound to the capture antibody(ies).

In some embodiments, the quantified amount of bound cardiac troponin(s) is compared to a reference standard. In some embodiments, the quantified amount of bound cardiac troponins comprises the amount of bound cardiac troponins of a single type (e.g., bound cardiac Troponin T, bound cardiac Troponin I, or bound cardiac Troponin C). In other embodiments, the quantified amount of bound cardiac troponins comprises the amount of bound cardiac troponins of two types (e.g., any two of: bound cardiac Troponin T, bound cardiac Troponin I, and bound cardiac Troponin C). In other embodiments, the quantified amount of bound cardiac troponins comprises the amount of bound cardiac troponins of all types (e.g., bound cardiac Troponin T, bound cardiac Troponin I, and bound cardiac Troponin C).

In some embodiments, the reference range or reference value is derived from an empirical study from a reference population. In some embodiments, the reference value for cardiac Troponin T is 0.1 ng/mL. In some embodiments, the reference value for cardiac Troponin I is 0.07 ng/mL. In some embodiments, the reference value for creatine kinase MB isoenzyme (CKMB) is 10 ng/mL. In some embodiments, the reference value of myoglobin is 170 ng/mL.

In some embodiments, a risk of a cardiovascular disease (e.g., cardiodiabetes) is determined from the comparison between the quantified amount of bound cardiac troponin(s) and the reference range.

In some embodiments, the present disclosure provides systems and methods suitable for assessing a risk of developing a cardiac disease or disorder (e.g., ACS/MI/HF/stable angina/CHD) comprising detecting a level of at least one cardiac Troponin In a subject having a normal level of another biomarker associated with the cardiac disease or disorder.

In some embodiments, the present disclosure provides systems and methods suitable for assessing a risk of developing a cardiac disease or disorder in a subject that is asymptomatic for the cardiac disease or disorder, that has an unknown CAD status, and/or that has a stable CAD (e.g., not vulnerable plaque unstable CAD).

In some embodiments, the biological sample is obtained from a subject in a non-acute-care settings (e.g., when the subject is asymptomatic for the cardiac disease or disorder).

In some embodiments, the method comprises monitoring a subject receiving treatment for a cardiac disease or disorder.

In some embodiments, the method comprises monitoring a subject's compliance with a therapeutic regimen for a cardiac disease or disorder. In some embodiments, the therapeutic regimen commonly causes elevated troponins. In some embodiments, the method further comprises recommending a change in the therapeutic regimen (e.g., a change in dose, a change in dosing frequency, a discontinuance of the therapeutic regimen, or a replacement of the drug(s) of the therapeutic regimen with alternate drug(s)) if a quantified amount of bound cardiac troponin(s) associated with a second biological sample from the subject is significantly greater than a quantified amount of bound cardiac troponin(s) associated with a first biological sample (e.g., a biological sample obtained from the subject before the second biological sample, such as before the therapeutic regimen was initiated).

In some embodiments, the method comprises identifying a subject as having or as being at risk for developing a cardiac disease or disorder, wherein the subject has tested negative for other biomarkers of the cardiac disease or disorder. In some embodiments, the cardiac disease or disorder is MUMS, and the other biomarkers comprise NT-pro BNP.

Limitations and differences of concentration measurements as a number of logs above level of detection (LOD) and/or LOQ. LOQ is the reliable level of detection (10% or less CV). In various embodiments, assays measure to 0.01, 0.001, 0.0001 micrograms or other very low concentrations using highly sensitive instrumentation. The concentrations are measured for a baseline and/or baseline AND fluctuations within at most 1 log above LOQ, at most 2 logs over LOQ, at most 3 logs over LOQ, at most 4 logs over LOQ.

In some embodiments, the reference values or ranges comprise a combination of troponins. In one embodiment the standards are comprised of components from a biological source. In some embodiments, a reference standard comprises a combination of troponins derived from (e.g., harvested from) cultured human cardiomyocytes In other embodiments the reference standards are synthetically produced or from cell culture.

In other embodiments, the method uses pull-down antibodies including polyclonals, or antibodies that recognize an epitope common to both cardiac Troponin T and cardiac Troponin I.

In some embodiments, the present disclosure provides a method of treating or preventing a cardiovascular disease or disorder in a subject. In some embodiments, the method comprises determining a cardiac Troponin I level and a cardiac Troponin T level in a biological sample of the subject; calculating an combined score as a function of at least the determined cardiac Troponin I level and the determined cardiac Troponin T level; comparing the combined score to reference values from a reference population; and initiating a treatment regimen in the subject if the combined score correlates with a range in a higher unit of an ordered distribution of the reference values. In some embodiments, the cardiovascular disease or disorder is an ischemic event. In some embodiments, the method comprises preventing a cardiovascular morbidity. In some embodiments, the cardiovascular disease or disorder is myocardial damage. In some embodiments, the step of determining the cardiac Troponin I and cardiac Troponin T levels comprises contacting the biological sample with an antibody that binds to both cardiac Troponin I and cardiac Troponin T. In some embodiments, the step of calculating the combined score consists essentially of adding the cardiac Troponin I level and the cardiac Troponin T level. In some embodiments, the method further comprises determining a second cardiac Troponin I level and a second cardiac Troponin T level in a second biological sample of the subject; calculating a second combined score as a function of at least the second cardiac Troponin I level and a second cardiac Troponin T level; comparing the second combined score to the combined core; and modifying the treatment regimen in the subject if the second combined score is higher or lower than the combined score. In some embodiments, the step of determining the cardiac Troponin I and cardiac Troponin T levels comprises contacting the biological sample with a monoclonal antibody that binds to both cardiac Troponin I and cardiac Troponin T to a microtiter well or to magnetic or latex beads. In some embodiments, the method further comprises determining a level of at least one additional biomarker in the biological sample, wherein the treatment regimen is initiated if the level of the at least one additional biomarker is outside a normal range for that biomarker. In some embodiments, the additional biomarker is selected from the group consisting of: cardiac muscle injury, ischemia, ischemia-reperfusion injury, fibrosis, apoptosis, cardiomyopathy, inflammation, complement cascade, glycemic control, insulin resistance, beta cell dysfunction, exercise physiology, diet, cerebrovascular events, hypertension, kidney function, and cachexia. In some embodiments, the method further comprises additional markers as part of a composite index score. In some embodiments, the step of determining the cardiac Troponin I level and the cardiac Troponin T level comprises contacting a solid surface with the biological sample, wherein the solid surface comprises at least one capture antibody capable of binding to cardiac Troponin T and at least one capture antibody capable of binding to cardiac Troponin I. In some embodiments, the at least one capture antibody capable of binding to cardiac Troponin T is capable of binding to whole free cardiac Troponin T, a proteolytic fragment of cardiac Troponin T, a post-translational modification of cardiac Troponin T, and/or a complex of cardiac Troponin T. In some embodiments, the at least one capture antibody capable of binding to cardiac Troponin I is capable of binding to whole free cardiac Troponin I, an epitope of cardiac Troponin I that is exposed when it is bound in complex to cardiac Troponin C, an epitope of cardiac Troponin C that is exposed when it is bound in complex to cardiac Troponin I, an epitope unique to a bound cardiac Troponin I:cardiac Troponin C complex, a proteolytic fragment of cardiac Troponin I, and/or a post-translational modification of cardiac Troponin I. In some embodiments, the solid surface further comprises at least one secondary antibody, wherein the at least one secondary antibody is capable of binding to a distinct epitope of whole free cardiac Troponin T, a proteolytic fragment of cardiac Troponin T, a post-translational modification of cardiac Troponin T, and/or a complex of cardiac Troponin T compared to an epitope that binds to the at least one capture antibody capable of binding to cardiac Troponin T; and at least one additional secondary antibody, wherein the at least one additional secondary antibody is capable of binding to a distinct epitope of whole free cardiac Troponin I, an epitope of cardiac Troponin I that is exposed when it is bound in complex to cardiac Troponin C, an epitope of cardiac Troponin C that is exposed when it is bound in complex to cardiac Troponin I, an epitope unique to a bound cardiac Troponin I:cardiac Troponin C complex, a proteolytic fragment of cardiac Troponin I, and/or a post-translational modification of cardiac Troponin I compared to an epitope that binds to the at least one capture antibody capable of binding to cardiac Troponin C. In some embodiments, the at least one capture antibody capable of binding to cardiac Troponin T and at least one capture antibody capable of binding to cardiac Troponin I comprise a polyclonal mixture. In some embodiments, the method further comprises contacting the biological sample with secondary detection antibodies of identically labelled polyclonal mixtures, wherein the secondary detection antibodies are specific for forms of cardiac Troponin T; and contacting the biological sample with at least one polyclonal mixture specific to at least cardiac Troponin I, cardiac Troponin C, or both. In some embodiments, the solid surface further comprises secondary detection antibodies of identically labelled polyclonal mixtures, wherein the secondary detection antibodies are specific for forms of cardiac Troponin T; and contacting the biological sample with at least one polyclonal mixture specific to at least cardiac Troponin I, cardiac Troponin C, or both. In some embodiments, the subject is asymptomatic for acute cardiovascular disease. In some embodiments, the subject is negative for biomarkers comprising cardiovascular stress and damage.

In some embodiments, the present disclosure provides a solid surface comprising at least one capture antibody capable of binding to cardiac Troponin T and at least one capture antibody capable of binding to cardiac Troponin I. In some embodiments, the at least one capture antibody capable of binding to cardiac Troponin T is capable of binding to whole free cardiac Troponin T, a proteolytic fragment of cardiac Troponin T, a post-translational modification of cardiac Troponin T, and/or a complex of cardiac Troponin T. In some embodiments, the at least one capture antibody capable of binding to cardiac Troponin I is capable of binding to whole free cardiac Troponin I, an epitope of cardiac Troponin I that is exposed when it is bound in complex to cardiac Troponin C, an epitope of cardiac Troponin C that is exposed when it is bound in complex to cardiac Troponin I, an epitope unique to a bound cardiac Troponin I:cardiac Troponin C complex, a proteolytic fragment of cardiac Troponin I, and/or a post-translational modification of cardiac Troponin I. In some embodiments, the solid surface further comprises at least one secondary antibody, wherein the at least one secondary antibody is capable of binding to a distinct epitope of whole free cardiac Troponin T, a proteolytic fragment of cardiac Troponin T, a post-translational modification of cardiac Troponin T, and/or a complex of cardiac Troponin T compared to an epitope that binds to the at least one capture antibody capable of binding to cardiac Troponin T; and at least one additional secondary antibody, wherein the at least one additional secondary antibody is capable of binding to a distinct epitope of whole free cardiac Troponin I, an epitope of cardiac Troponin I that is exposed when it is bound in complex to cardiac Troponin C, an epitope of cardiac Troponin C that is exposed when it is bound in complex to cardiac Troponin I, an epitope unique to a bound cardiac Troponin I:cardiac Troponin C complex, a proteolytic fragment of cardiac Troponin I, and/or a post-translational modification of cardiac Troponin I compared to an epitope that binds to the at least one capture antibody capable of binding to cardiac Troponin C. In some embodiments, the at least one capture antibody capable of binding to cardiac Troponin T and at least one capture antibody capable of binding to cardiac Troponin I comprise a polyclonal mixture. In some embodiments, the solid surface further comprises secondary detection antibodies of identically labelled polyclonal mixtures, wherein the secondary detection antibodies are specific for forms of cardiac Troponin T; and contacting the biological sample with at least one polyclonal mixture specific to at least cardiac Troponin I, cardiac Troponin C, or both.

1. A method of treating or preventing a cardiovascular disease or disorder in a subject, the method comprising:
determining a cardiac Troponin I level and a cardiac Troponin T level in a biological sample of the subject;
calculating an combined score as a function of at least the determined cardiac Troponin I level and the determined cardiac Troponin T level;
comparing the combined score to reference values from a reference population; and
initiating a treatment regimen in the subject if the combined score correlates with a range in a higher unit of an ordered distribution of the reference values.

2. The method of claim 1, wherein the cardiovascular disease or disorder is an ischemic event.

3. The method of claim 1, wherein the method comprises preventing a cardiovascular morbidity.

4. The method of claim 1, wherein the cardiovascular disease or disorder is myocardial damage.

5. The method of claim 1, wherein the step of determining the cardiac Troponin I and cardiac Troponin T levels comprises contacting the biological sample with an antibody that binds to both cardiac Troponin I and cardiac Troponin T.

6. The method of claim 1, wherein the step of calculating the combined score consists essentially of adding the cardiac Troponin I level and the cardiac Troponin T level.

7. The method of claim 1 further comprising:
determining a second cardiac Troponin I level and a second cardiac Troponin T level in a second biological sample of the subject;
calculating a second combined score as a function of at least the second cardiac Troponin I level and a second cardiac Troponin T level;
comparing the second combined score to the combined core; and
modifying the treatment regimen in the subject if the second combined score is higher or lower than the combined score.

8. The method of claim 1, wherein the step of determining the cardiac Troponin I and cardiac Troponin T levels comprises contacting the biological sample with a monoclonal antibody that binds to both cardiac Troponin I and cardiac Troponin T to a microtiter well or to magnetic or latex beads.

9. The method of claim 1 further comprising determining a level of at least one additional biomarker in the biological sample, wherein the treatment regimen is initiated if the level of the at least one additional biomarker is outside a normal range for that biomarker.

10. The method of claim 9 wherein the additional biomarker is selected from the group consisting of: cardiac muscle injury, ischemia, ischemia-reperfusion injury, fibrosis, apoptosis, cardiomyopathy, inflammation, complement cascade, glycemic control, insulin resistance, beta cell dysfunction, exercise physiology, diet, cerebrovascular events, hypertension, kidney function, and cachexia.

11. The method of any of claims 1-4, further comprising additional markers as part of a composite index score.

12. The method of claim 1, wherein the step of determining the cardiac Troponin I level and the cardiac Troponin T level comprises contacting a solid surface with the biological sample, wherein the solid surface comprises at least one capture antibody capable of binding to cardiac Troponin T and at least one capture antibody capable of binding to cardiac Troponin I.

13. The method of claim 12, wherein the at least one capture antibody capable of binding to cardiac Troponin T is capable of binding to whole free cardiac Troponin T, a proteolytic fragment of cardiac Troponin T, a post-translational modification of cardiac Troponin T, and/or a complex of cardiac Troponin T.

14. The method of claim 12, wherein the at least one capture antibody capable of binding to cardiac Troponin I is capable of binding to whole free cardiac Troponin I, an epitope of cardiac Troponin I that is exposed when it is bound in complex to cardiac Troponin C, an epitope of cardiac Troponin C that is exposed when it is bound in complex to cardiac Troponin I, an epitope unique to a bound cardiac Troponin I:cardiac Troponin C complex, a proteolytic fragment of cardiac Troponin I, and/or a post-translational modification of cardiac Troponin I.

15. The method of claim 14, wherein the solid surface further comprises:
at least one secondary antibody, wherein the at least one secondary antibody is capable of binding to a distinct epitope of whole free cardiac Troponin T, a proteolytic fragment of cardiac Troponin T, a post-translational modification of cardiac Troponin T, and/or a complex of cardiac Troponin T compared to an epitope that binds to the at least one capture antibody capable of binding to cardiac Troponin T; and
at least one additional secondary antibody, wherein the at least one additional secondary antibody is capable of binding to a distinct epitope of whole free cardiac Troponin I, an epitope of cardiac Troponin I that is exposed when it is bound in complex to cardiac Troponin C, an epitope of cardiac Troponin C that is exposed when it is bound in complex to cardiac Troponin I, an epitope unique to a bound cardiac Troponin I:cardiac Troponin C complex, a proteolytic fragment of cardiac Troponin I, and/or a post-translational modification of cardiac Troponin I compared to an epitope that binds to the at least one capture antibody capable of binding to cardiac Troponin C.

16. The method of claim 12 wherein the at least one capture antibody capable of binding to cardiac Troponin T and at least one capture antibody capable of binding to cardiac Troponin I comprise a polyclonal mixture.

17. The method of claim 5 further comprising contacting the biological sample with:
secondary detection antibodies of identically labelled polyclonal mixtures, wherein the secondary detection antibodies are specific for forms of cardiac Troponin T; and
contacting the biological sample with at least one polyclonal mixture specific to at least cardiac Troponin I, cardiac Troponin C, or both.

18. The method of claim 12, wherein the solid surface further comprises:
secondary detection antibodies of identically labelled polyclonal mixtures, wherein the secondary detection antibodies are specific for forms of cardiac Troponin T; and
contacting the biological sample with at least one polyclonal mixture specific to at least cardiac Troponin I, cardiac Troponin C, or both.

19. The method of claim 1, wherein the subject is asymptomatic for acute cardiovascular disease.

20. The method of claim 1, wherein the subject is negative for biomarkers comprising cardiovascular stress and damage.
